(19) United States
(12) Patent Application Publication
(10) Pub. No.: US 2025/0115861 A1
(43) Pub. Date: Apr. 10, 2025

(54) CELL HANDLING SYSTEM, CELL HANDLING DEVICE, AND CELL HANDLING CONTROL APPARATUS

(71) Applicants: Canon Medical Systems Corporation, Otawara-shi (JP); CANON KABUSHIKI KAISHA, Tokyo (JP)

(72) Inventor: Shinya SUGIYAMA, Nasushiobara (JP)

(73) Assignees: Canon Medical Systems Corporation, Otawara-shi (JP); CANON KABUSHIKI KAISHA, Tokyo (JP)

(21) Appl. No.: 18/905,947

(22) Filed: Oct. 3, 2024

(30) Foreign Application Priority Data
Oct. 6, 2023 (JP) 2023-174690

(51) Int. Cl. C12M 1/36

(52) U.S. Cl. CPC C12M 41/48

(57) ABSTRACT

A cell handling system includes a cell handling device and a cell handling control apparatus. The cell handling device is configured so that cell handling is performed inside the cell handling device. The cell handling device includes an information presenter configured to present identification information for identifying a process performed in the cell handling device. The cell handling control apparatus is configured to control the cell handling performed in the cell handling device. The cell handling control apparatus includes processing circuitry configured to acquire the identification information presented by the information presenter and determine a parameter relating to control of the cell handling based on the acquired identification information.

### CROSS-REFERENCE TO RELATED APPLICATIONS

This application is based upon and claims the benefit of priority from Japanese Patent Application No. 2023-174690, filed Oct. 6, 2023, the entire contents of which are incorporated herein by reference.

### FIELD

Embodiments described herein relate generally to a cell handling system, a cell handling device, and a cell handling control apparatus.

### BACKGROUND

There is a cell handling system that automatically performs cell handling which includes both cell processing and culturing. The cell handling system is used, for example, to produce an induced pluripotent stem cell (hereinafter referred to as an “iPS cell”).

In order to secure a sterile condition of such a cell handling system, a closed cell handling device is used for performing cell handling. Also, it is convenient if the user can use different cell handling devices that perform different types of cell handling processes according to the specific type or purpose of cell handling.

To prevent contamination (interfusion), a cell handling device is preferably discarded after use-without being reused. Thus, installing a supply source for supplying energy, gas, a reagent, or the like that is used for cell handling inside the cell handling device results in increased costs. Because of this, a supply source for supplying energy, gas, a reagent, or the like that is used for cell handling, and a control unit for controlling cell handling in a cell handling device are preferably arranged outside the cell handling device and subsequently reused. An energy source is, for example, a drive unit for supplying power, a heater for performing temperature control, or the like. In this case, since the timing of a reagent flowing in and the time period for performing temperature control, etc., vary according to the type of cell handling, a control unit needs to perform control through making use of a control parameter according to the type of cell handling device.

### BRIEF DESCRIPTION OF THE DRAWINGS

FIG. 1 is a diagram showing a configuration of a cell handling system according to a first embodiment.

FIG. 2 is a diagram for explaining a flow of data in the cell handling system according to the first embodiment.

FIG. 3 is a flowchart illustrating a control processing procedure performed by the system according to the first embodiment.

FIG. 4 is a diagram for explaining a control method adopted in the case of performing cell processing and culturing using a cell handling device.

FIG. 5 is a diagram for explaining a control method adopted in the case of performing culturing using a cell handling device.

FIG. 6 is a diagram showing a configuration of a cell handling system according to a second embodiment.

FIG. 7 is a diagram showing a configuration of a cell handling system according to a third embodiment.

### DETAILED DESCRIPTION

In general, according to one embodiment, a cell handling system includes a cell handling device and a cell handling control apparatus. The cell handling device is configured so that cell handling is performed inside the cell handling device. The cell handling device includes an information presenter configured to present identification information for identifying a process performed in the cell handling device. The cell handling control apparatus is configured to control the cell handling performed in the cell handling device. The cell handling control apparatus includes processing circuitry configured to acquire the identification information presented by the information presenter and determine a parameter relating to control of the cell handling based on the acquired identification information.

Hereinafter, a cell handling system will be described with reference to the accompanying drawings. In the description below, constituents having substantially the same functions and configurations will be denoted by the same reference symbols, and a repeat description of such constituents will be given only where necessary.

### First Embodiment

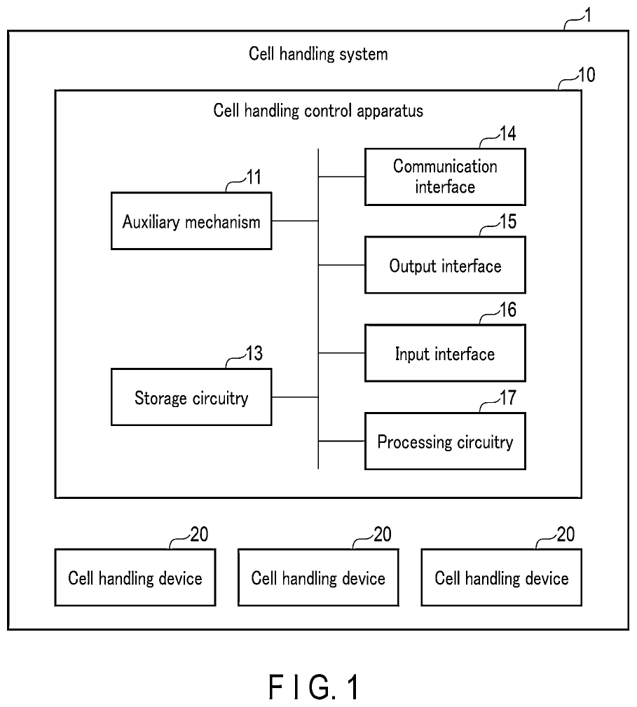

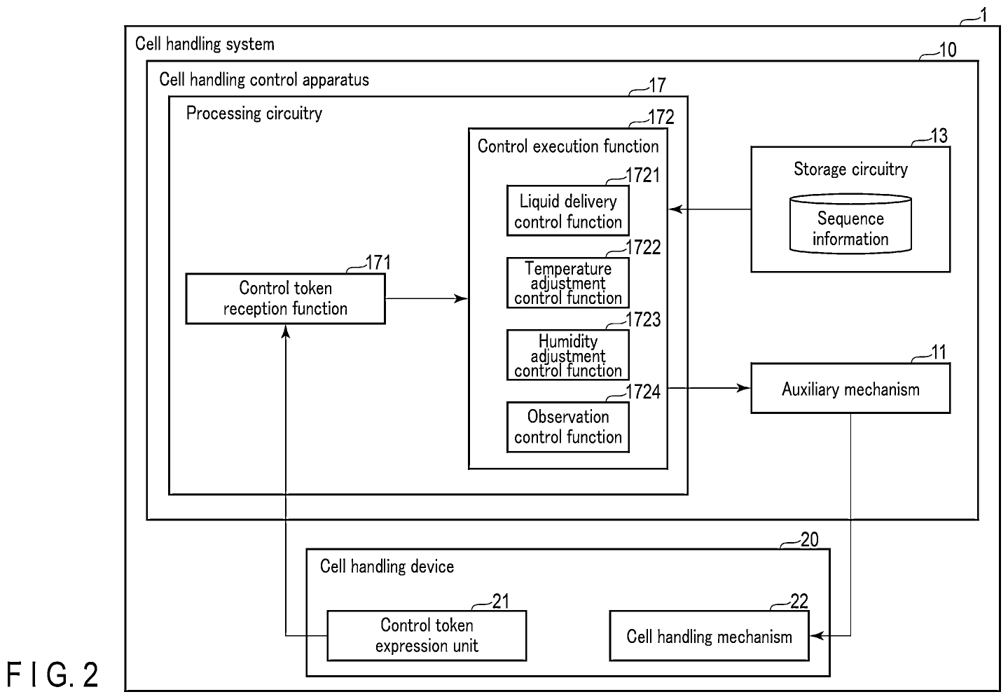

FIG. 1 is a block diagram showing an example of a configuration of a cell handling system 1 according to a first embodiment. FIG. 2 is a diagram for explaining a flow of data in the cell handling system 1. The cell handling system 1 is a system for automatically performing a cell handling process. The cell handling system 1 is used to, for example, produce an iPS cell. The cell handling system 1 can be used not only to produce an iPS cell but also to produce or process any cells.

As shown in FIG. 1, the cell handling system 1 includes a cell handling control apparatus 10 and a cell handling device 20. The cell handling device 20 is a device inside which cell handling is performed, and is attached to the cell handling control apparatus 10 in a detachable fashion. The cell handling control apparatus 10 controls the cell handling performed in the cell handling device 20.

The cell handling device 20 is, for example, a closed cartridge inside which cell handling is performed. The cell handling includes cell processing and/or cell culturing. The cell handling process includes a process of processing a cell (hereinafter referred to as a “cell processing process”) and/or a process of culturing a cell (hereinafter referred to as a “cell culturing process”). The cell processing process includes, for example, a process of separating blood cells in the blood to extract necessary cells or a process of introducing a factor into a peripheral blood mononuclear cell (hereinafter referred to a “PBMC”) after expansion culture. The cell handling device 20 may perform all of the above processes or only some of the above processes. The cell handling device 20 may be referred to as a “processing culturing device”.

After performing a predetermined cell handling, the cell handling device 20 is discarded to prevent infection. Multiple types of cell handling devices 20 that perform different processes can be replaced with each other and attached to the cell handling control apparatus 10. For example, cell handling devices 20 that perform different types of processes such as a cell processing process and a cell culturing process can be replaced with each other and attached to an attachment portion of the cell handling control apparatus 10. Also, cell handling devices 20 that perform the same type of process under different conditions of that process may be replaced with each other and attached to the cell handling control apparatus 10. The conditions of the process are, for example, the type of cell fluid or reagent placed in the cell handling device 20, the amount of liquid, the speed of injection, etc. The cell handling system 1 shown in FIG. 1 is a system that is simultaneously provided with three cell handling devices 20 and is capable of simultaneously performing cell handling in each cell handling device 20. It suffices that the cell handling system 1 allows one or more cell handling devices 20 to be attached thereto, and the cell handling system 1 may allow four or more cell handling devices 20 to be attached thereto.

The cell handling device 20 includes a control token expression unit 21 and a cell handling mechanism 22 that performs a cell handling process.

The cell handling mechanism 22 performs a preset cell handling. The cell handling mechanism 22 includes one or more closed passages and containers inside the cell handling mechanism 22. Typically, the cell handling device 20 includes multiple passages and multiple containers inside the cell handling device 20. Each passage is a pipeline for conveying a liquid including a reagent or a cell used to handle a cell, and is formed of a tube-shaped resin or plastic. Each container is formed of a resin or plastic. For example, polycarbonate (PC) or polystyrene (PS) is used as a resin. The liquid may be blood, a blood suspension, a liquid containing PBMCs, a liquid containing cells such as a culture liquid, or a liquid used for cell processing such as a reagent, medium, or factor.

The control token expression unit 21 causes a control token to be expressed. The control token is a token that includes identification information for identifying the process performed by the cell handling mechanism 22. The control token is, for example, realized by a numerical value, a symbol, a character, a mark, an image, an IC lock, a component shape, a light, a sound, etc. The control token is, for example, a model number, a serial number, etc., that shows the type of cell handling device 20. If different types of liquid or gas are used for the cell handling according to the type of cell handling device 20, the type of liquid or the type of gas may be used as the control token. The amount of liquid or gas used for the cell handling may also be used as the control token. A combination of the aforementioned elements may also be used as the control token.

The control token expression unit 21 presents the control token that includes identification information to the cell handling control apparatus 10. The control token expression unit 21 is an example of an information presenter that presents identification information. The control token expression unit 21 is, for example, an image that shows a model number or a serial number, or a bar-code or a mark that shows the type of cell handling device 20. The control token expression unit 21 may also be a connector having a shape corresponding to the type of cell handling device 20. For example, in this case, the shape of a protrusion of the connector is formed to correspond to the type of cell handling device 20. The control token expression unit 21 may also be a device that outputs a voice that expresses the process performed in the cell handling device 20, or a light-emitting device that emits a light-emitting pattern or color that shows the process performed in the cell handling device 20. The control token expression unit 21 may be a communication interface capable of communicating the data showing the process performed in the cell handling device 20 to the cell handling control apparatus 10, either wirelessly or via a wire.

Next, a configuration of the cell handling control apparatus 10 will be described.

The cell handling control apparatus 10 includes an auxiliary mechanism 11, storage circuitry 13, a communication interface 14, an output interface 15, an input interface 16, and processing circuitry 17. The cell handling control apparatus 10 may be referred to as a “control unit” that controls the cell handling performed in the cell handling device 20.

The auxiliary mechanism 11 includes one or more devices that assist the cell handling performed inside the cell handling device 20. The auxiliary mechanism 11 supplies, for example, energy for the performance of the cell handling, or a reagent or gas used for the cell handling. The auxiliary mechanism 11 is, for example, a liquid delivery mechanism such as a cylinder or a pneumatic device, a temperature adjustment mechanism such as a heater, a humidity adjustment mechanism for adjusting humidity, an observation mechanism such as a camera. The auxiliary mechanism 11 may be a cylinder for supplying a reagent or gas from the outside, a holding mechanism for adjusting the position or inclination of the cell handling device 20, a shaking mechanism for shaking the cell handling device 20, a power source for supplying power to each of the aforementioned mechanisms, etc. The auxiliary mechanism 11 may be a mechanism for supplying a magnetic force to each of the aforementioned mechanisms in the form of drive energy, or a magnet for opening or closing a valve provided in the passage.

The storage circuitry 13 stores programs to be executed by the processing circuitry 17, various types of data to be used in the processing performed by the processing circuitry 17, and the like. Such programs include, for example, a program that is pre-installed in a computer via a network or a non-transitory computer-readable storage medium to cause the computer to implement each function of the processing circuitry 17. The various types of data as used herein are typically digital data. The storage circuitry 13 is an example of a storage.

Also, the storage circuitry 13 stores information relating to a sequence for performing cell handling (hereinafter referred to as “sequence information”). The sequence information includes control parameters for controlling operation of each device of the auxiliary mechanism 11 in the cell handling. The sequence information also includes the time-series information of each control parameter. For example, the sequence information includes the operation timing of each device of the auxiliary mechanism 11, the order of the operations, etc. The storage circuitry 13 stores a sequence according to the type or conditions of the process performed in the cell handling device 20. The storage circuitry 13 stores sequence information for each type of cell handling device 20. In this case, the storage circuitry 13 stores, for example, a sequence for the case of using a cell handling device 20 that performs a cell processing process; a sequence for the case of using a cell handling device 20 that performs a cell culturing process; and a sequence for the case of using a cell handling device 20 that performs both a cell processing process and a cell culturing process. Also, even if the same process is to be performed, different sequences may be stored for each reagent to be used as the sequences for the case where the process has different conditions.

The communication interface 14 is an interface for performing data communication with an external device via network.

The output interface 15 is connected to the processing circuitry 17 and outputs a signal supplied from the processing circuitry 17. The output interface 15 is realized, for example, by display circuitry, print circuitry, a voice device, and the like. The display circuitry includes, for example, a CRT display, a liquid crystal display, an organic EL display, an LED display, a plasma display, and the like. The display circuitry may also be processing circuitry that converts data showing a display target into a video signal and outputs the video signal to an external device. The print circuitry includes, for example, a printer and the like. The print circuitry may also be output circuitry that outputs data showing a print target to an external device. The voice device includes, for example, a speaker and the like. The voice device may be output circuitry that outputs a voice signal to an external device.

The input interface 16 receives, for example, various operations from an operator. The input interface 16 is realized by, for example, a mouse, a keyboard, a touch pad which allows input of instructions through a touch on its operation screen, etc. The input interface 16 is connected to the processing circuitry 17, thereby converting an operational command that is input by the operator into an electric signal and outputting the electric signal to the processing circuitry 17.

The input interface 16 is not limited to one that includes physical operational components such as a mouse and a keyboard. For example, the input interface 16 may be processing circuitry that receives an electric signal corresponding to an operation command input from an external input device provided separately from the cell handling system 1 and outputs the electric signal to the processing circuitry 17.

The processing circuitry 17 is a processor that functions as the center of the cell handling system 1. As shown in FIG. 2, the processing circuitry 17 comprehensively controls each component of the cell handling system 1 by executing a program read from the storage circuitry 13. Also, the processing circuitry 17 performs a control token reception function 171 and a control execution function 172 by executing a program read from the storage circuitry 13. The processing circuitry 17 is an example of a controller.

With the control token reception function 171, the processing circuitry 17 receives the input of a control token from the cell handling device 20 attached to the cell handling control apparatus 10. With the control token reception function 171, the processing circuitry 17 acquires identification information included in the control token presented by the control token expression unit 21. The processing circuitry 17 that implements the control token reception function 171 is an example of an identification information acquisition unit. The processing circuitry 17 that implements the control token reception function 171 may be referred to as a “control token receiver”.

For example, if an image attached to the exterior of the cell handling device 20 is used as the control token expression unit 21, a camera for capturing the image is provided to the cell handling control apparatus 10. The processing circuitry 17 reads a mark or a number shown on the image as a control token by performing an image analysis on the image captured by the camera. If a light-emitting device is used as the control token expression unit 21, a sensor capable of detecting a light from the light-emitting device is provided to the cell handling control apparatus 10. The processing circuitry 17 analyzes the control token by analyzing the color, pattern, spectrum, etc., of the light detected by the sensor. If a protrusion having a specific shape and provided to a connector is used as the control token expression unit 21, a camera or a pressure sensor capable of detecting the shape of the protrusion is provided to a connector on the cell handling control apparatus 10 side. The processing circuitry 17 acquires the control token by specifying the detected shape of the protrusion. If a communication interface capable of transmitting identification information is used as the control token expression unit 21, the processing circuitry 17 acquires the identification information received at the communication interface 14 as the control token.

With the control execution function 172, the processing circuitry 17 determines a parameter relating to cell handling control based on the identification information included in the acquired control token. For example, with the control execution function 172, the processing circuitry 17 executes cell handling control inside the installed cell handling device 20 by determining a sequence to be performed based on the control token, determining a control parameter according to the determined sequence, and executing the control by the control execution function 172 based on the determined control parameter. At this time, the processing circuitry 17 determines the process to be performed by the attached cell handling device 20 based on the acquired control token, reads, from among multiple sequences stores in the storage circuitry 13, a sequence corresponding to the determination result and controls the operation of the auxiliary mechanism 11 based on the read sequence. Also, at this time, the processing circuitry 17 performs, on the auxiliary mechanism 11, a direct control for directly controlling the drive, a period control for controlling a period, and an information acquisition control for causing information to be acquired. The processing circuitry 17 that implements the control execution function 172 is an example of a control determination unit. The processing circuitry 17 that implements the control execution function 172 may be referred to as an example of a control execution unit.

Here, the control execution function 172 will be described in more detail. The control execution function 172 has a liquid delivery control function 1721, a temperature adjustment control function 1722, a humidity adjustment control function 1723, and an observation control function 1724.

With the liquid delivery control function 1721, the processing circuitry 17 controls the processes such as the injection of a cell, the injection of a reagent, the substitution of a liquid such as a medium, and the collection of cells, by controlling the drive of a cylinder, a pump, an open-close valve, etc., based on a sequence. At this time, various timings, the cell type, the type of liquid such as a reagent, the liquid flow rate, the total amount of liquid, the position or angle of a container, etc., are used as control parameters. The processing circuitry 17 that implements the liquid delivery control function 1721 is an example of a liquid delivery controller.

With the temperature adjustment control function 1722, the processing circuitry 17 controls the processes such as temperature adjustment performed when factor introduction or culturing is performed, by controlling the drive of a heater or the operation of a power source that supplies power to the heater. At this time, a target temperature, a temperature adjustment period, the timing of commencing and ending temperature adjustment, the temperature of a heater, the power supplied to the heater, etc., are used as control parameters. The processing circuitry 17 that implements the temperature adjustment control function 1722 is an example of a temperature adjustment controller.

With the humidity adjustment control function 1723, the processing circuitry 17 controls the processes such as humidity adjustment performed when factor introduction or culturing is performed, by controlling the drive of a mechanism that adjusts humidity based on a sequence. At this time, a target humidity, a humidity adjustment period, the timing of commencing and ending humidity adjustment, etc., are used as control parameters. The processing circuitry 17 that implements the humidity adjustment control function 1723 is an example of a humidity adjustment controller.

With the observation control function 1724, the processing circuitry 17 controls the process of observing a cell when factor introduction or culturing is performed, by controlling the operation of an observation device such as a camera or sensor or the operation of a movement device for moving an observation device based on a sequence. At this time, an observation time period, the timing of commencing and ending observation, the position of an observation device, etc., are used as control parameters. The processing circuitry 17 that implements the observation control function 1724 is an example of an observation controller.

Next, an operation of the cell handling system 1 of the present embodiment will be described.

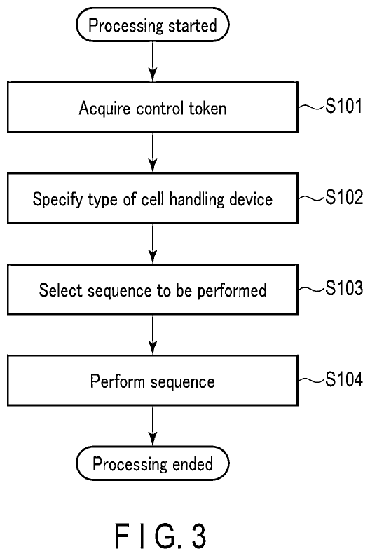

FIG. 3 is a flowchart showing an example of a procedure of sequence execution performed by the cell handling control apparatus 10 of the cell handling system 1. The sequence execution is the processing of controlling operation of each device of the auxiliary mechanism 11 according to a prestored sequence when performing cell handling using the cell handling device 20. The procedure of each step described below is a mere example, and each step can be altered as appropriate to the extent possible. Omission, replacement, and addition of a step can be made as appropriate in the procedure explained below according to the manner in which the embodiment will be implemented. The foregoing explanation of the procedure of each step applies to each of the embodiments and modifications described below.

In the case of performing cell handling using the cell handling device 20, first, a user attaches, to the cell handling control apparatus 10, a cell handling device 20 to be used.

### (Step S101)

First, by implementing the control token reception function 171, the processing circuitry 17 acquires a control token from the cell handling device 20. For example, if a bar-code into which information indicating the type of cell handling device 20 is built is used as the control token expression unit 21, the processing circuitry 17 controls an operation of a camera capable of capturing an image of a bar-code attached to the exterior of the cell handling device 20, and captures an image of the bar-code using the camera, whereby the processing circuitry 17 acquires a control token. The timing of acquiring a control token may be either when the cell handling device 20 is attached or at the timing of performing cell handling.

### (Step S102)

Next, by implementing the control execution function 172, the processing circuitry 17 specifies the type of cell handling device 20 based on the acquired control token. For example, if a bar-code into which information indicating the type of cell handling device 20 is built is used as the control token expression unit 21, the processing circuitry 17 reads the identification information included in the bar-code whose image has been captured by a camera, whereby the processing circuitry 17 specifies the type of cell handling device 20.

### (Step S103)

Next, by implementing the control execution function 172, the processing circuitry 17 selects a sequence to be performed based on the specified type of cell handling device 20. At this time, the processing circuitry 17 searches for, among multiple sequences prestored in the storage circuitry 13, a sequence corresponding to the type of the cell handling device 20 and reads the searched-for sequence.

### (Step S104)

Next, by implementing the control execution function 172, the processing circuitry 17 controls the cell handling performed using the cell handling device 20 according to the selected sequence. At this time, the processing circuitry 17 controls the operation of the auxiliary mechanism 11 at a predetermined timing using each control parameter included in the selected sequence, whereby the processing circuitry 17 controls the cell handling performed inside the cell handling device 20.

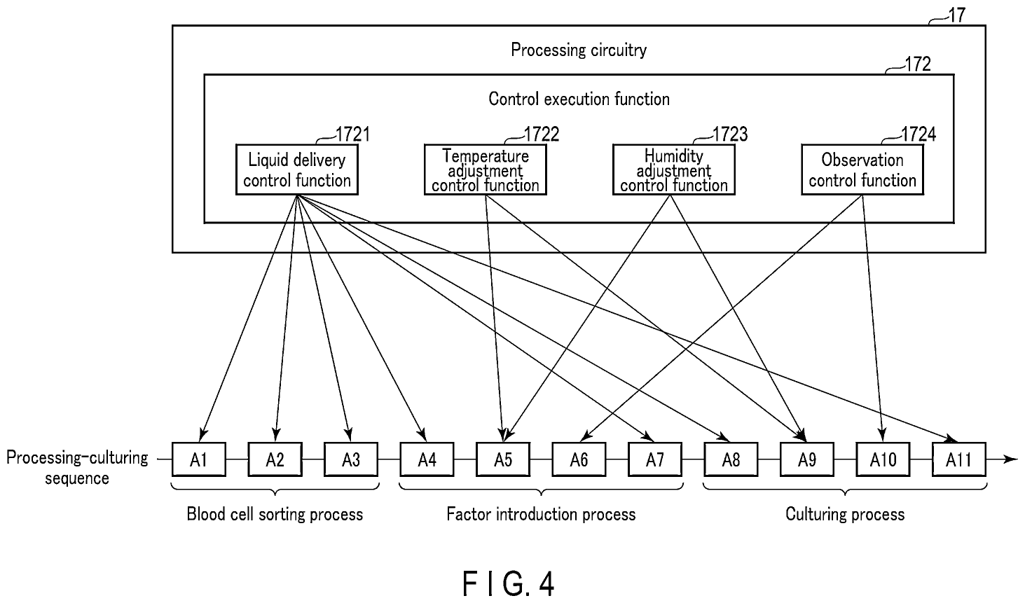

FIG. 4 is a diagram for explaining an example of the control performed by the control execution function 172 when a sequence corresponding to the cell handling device 20 that performs a cell processing process and a cell culturing process is performed. Omission, replacement, and addition of a process can be made as appropriate regarding the processes explained below according to the manner in which the embodiment will be implemented. Herein, a case of using a cell handling device 20 that performs cell processing and cell culturing for producing an iPS cell will be explained as an example.

As shown in FIG. 4, processes A1 through A11 are performed in the mentioned order in a sequence that uses a cell handling device 20 that performs a cell processing process and a cell culturing process. Processes A1 through A3 form a blood cell sorting process of separating blood cells contained in blood. For example, process A1 is a cell injection process of injecting a cell, process A2 is a reagent injection process of injecting a reagent, and process A3 is a liquid replacement process of replacing a liquid. Processes A4 through A7 form a factor introduction process of introducing a factor into a PBMC. For example, process A4 is a reagent injection process, process A5 is a temperature/humidity adjustment process of adjusting temperature and humidity, process A6 is an observation process of observing cells while the temperature/humidity adjustment process A5 is performed, and process A7 is a liquid replacement process of replacing a liquid such as a medium. Processes A8 through A11 form a culturing process of culturing a cell. Process A8 is a reagent injection process, process A9 is a temperature/humidity adjustment process, process A10 is an observation process of observing cells while the temperature/humidity adjustment process A9 is performed, and process A11 is a cell collection process of collecting cells.

In the case of performing the sequence shown in FIG. 4, by implementing the liquid delivery control function 1721, the processing circuitry 17 controls the drive of the auxiliary mechanism 11 in the cell injection process A1, the reagent injection process A2, the liquid replacement process A3, the reagent injection process A4, the liquid replacement process A7, the reagent injection process A8, and the cell collection process A11. By implementing the temperature adjustment control function 1722 and the humidity adjustment control function 1723, the processing circuitry 17 also controls the drive of the auxiliary mechanism 11 in the temperature/humidity adjustment process A5 and the temperature/humidity adjustment process A9. By implementing the observation control function 1724, the processing circuitry 17 also controls the drive of the auxiliary mechanism 11 in the observation process A6 and the observation process A10.

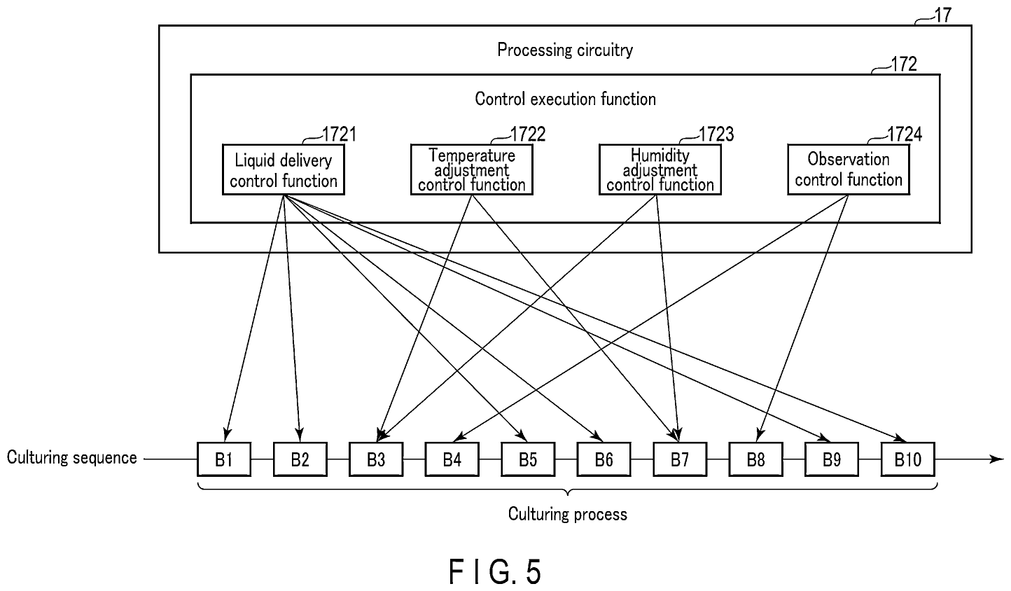

FIG. 5 is a diagram for explaining an example of the control performed by the control execution function 172 when a sequence corresponding to the cell handling device 20 that performs a cell culturing process is performed. Omission, replacement, and addition of a process can be made as appropriate regarding the processes explained below according to the manner in which the embodiment will be implemented. Herein, a case of using a cell handling device 20 that performs cell culturing for producing an iPS cell will be explained as an example.

As shown in FIG. 5, processes B1 through B10 are performed in the mentioned order in a sequence that uses a cell handling device 20 that performs a cell culturing process. Process B1 is cell injection process, process B2 is a liquid replacement process, process B3 is a temperature/humidity adjustment process, process B4 is an observation process of observing cells while the temperature/humidity adjustment process B3 is performed, process B5 is a liquid replacement process, process B6 is a liquid delivery process of delivering a liquid, process B7 is a temperature/humidity adjustment process, process B8 is an observation process of observing cells while the temperature/humidity adjustment process B7 is performed, process B9 is a liquid replacement process, and process B10 is a cell collection process.

In the case of performing the sequence shown in FIG. 5, by implementing the liquid delivery control function 1721, the processing circuitry 17 controls the drive of the auxiliary mechanism 11 in the cell injection process B1, the liquid replacement process B2, the liquid replacement process B5, the liquid delivery process B6, the liquid replacement process B9, and the cell collection process B10. By implementing the temperature adjustment control function 1722 and the humidity adjustment control function 1723, the processing circuitry 17 also controls the drive of the auxiliary mechanism 11 in the temperature/humidity adjustment process B3 and the temperature/humidity adjustment process B7. By implementing the observation control function 1724, the processing circuitry 17 also controls the drive of the auxiliary mechanism 11 in the observation process B4 and the observation process B8.

Hereinafter, the effects of the cell handling system 1 according to the present embodiment will be described.

The cell handling system 1 of the present embodiment includes the cell handling control apparatus 10, and the cell handling device 20 inside which cell handling is performed. Cell handling refers to, for example, cell processing and/or cell culturing. The cell handling control apparatus 10 controls the cell handling performed in the cell handling device 20. The cell handling device 20 includes the control token expression unit 21 as an information presenter. The control token expression unit 21 presents identification information for identifying the process performed inside the cell handling device 20. Also, the cell handling control apparatus 10 can acquire the identification information presented by the control token expression unit 21 and determine a parameter relating to the control of cell handling based on the acquired identification information.

For example, the cell handling control apparatus 10 can determine a parameter relating to the control of cell handling by determining a sequence that includes multiple processes based on the identification information. Specifically, the cell handling control apparatus 10 includes the storage circuitry 13 storing multiple sequences, and can specify the type of cell handling device 20 based on the identification information and then select a sequence corresponding to the specified type from among the sequences stored in the storage circuitry 13.

Also, the cell handling control apparatus 10 includes the auxiliary mechanism 11 for assisting the cell handling performed in the cell handling device 20, and can perform cell handling according to the determined sequence by controlling the operation of the auxiliary mechanism 11 based on the parameter relating to the determined control. The auxiliary mechanism 11 is a liquid delivery mechanism such as a cylinder or a pneumatic device, a temperature adjustment mechanism such as a heater, a humidity adjustment mechanism for adjusting humidity, an observation mechanism such as a camera. Also, the cell handling control apparatus 10 performs, on the auxiliary mechanism 11, a direct control for directly controlling drive, a period control for controlling a period, and an information acquisition control for causing information to be acquired.

Either disposal or maintenance is required for the cell handling device 20 that performs cell handling every time it is used in order to prevent infection. According to the cell handling system 1 of the present embodiment, since the auxiliary mechanism 11 for supplying drive force, power, thermal energy, a reagent, etc., for the performance of cell handling is provided in the cell handling control apparatus 10 that does not come into contact with cells, the production or maintenance costs of the cell handling system 1 can be reduced. Also, the cell handling device 20 can be configured in various and simple manners; thus, cell handling can be performed using a common cell handling control apparatus 10.

In the case of using the cell handling system 1 that can replace cell handling devices 20 that perform different processes, it is necessary to perform each control of the auxiliary mechanism 11 using a control parameter corresponding to the process to be performed by the attached cell handling device 20. According to the cell handling system 1 of the present embodiment, it is possible to automatically determine a sequence corresponding to the process to be performed based on the control token acquired from the cell handling device 20. Thus, it is possible to automatically determine an appropriate sequence corresponding to the type of process performed by the attached cell handling device 20 without requiring a user to determine a control parameter every time the user uses the cell handling device 20. This can save the user the trouble of determining and inputting a control parameter and thereby prevent incorrect input of a control parameter.

### (First Modification)

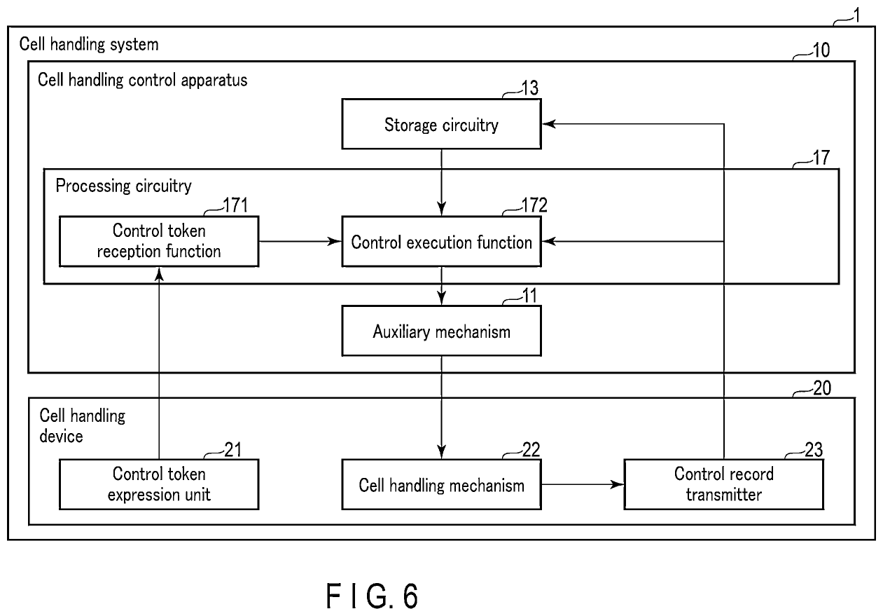

FIG. 6 is a diagram for explaining a flow of data in the cell handling system 1 according to a first modification. In the present modification, the cell handling device 20 includes a record transmitter 23, as shown in FIG. 6. The record transmitter 23 acquires execution information relating to the cell handling actually performed inside the cell handling device 20 and transmits the acquired execution information to the cell handling control apparatus 10. The execution information includes, for example, information relating to the result of executing the adjustment of a culturing environment and liquid delivery. For example, the record transmitter 23 is realized by a sensor for detecting the passing of a liquid, a sensor for detecting a culturing environment, or a communication interface for transmitting the result of the detection by the sensor.

The cell handling control apparatus 10 acquires the execution information transmitted from the record transmitter 23 and records the acquired execution information in the storage circuitry 13.

By implementing the control execution function 172, the processing circuitry 17 may sequentially acquire the execution information transmitted from the cell handling device 20 while cell handling is being performed and alter a control parameter according to the processing state and culturing state.

A sensor for detecting anomalies occurring inside the cell handling device 20 may be provided to the cell handling device 20. In this case, when acquiring a control token, the cell handling control apparatus 10 may acquire an anomaly detection result in addition to the control token.

### (Second Modification)

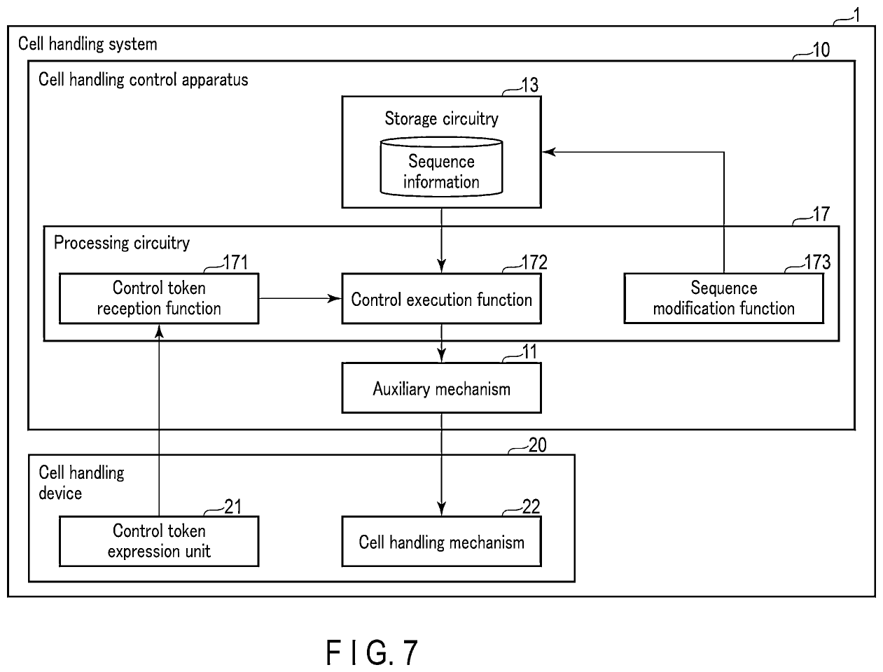

FIG. 7 is a diagram for explaining a flow of data in the cell handling system 1 according to a second modification. In the present modification, the processing circuitry 17 of the cell handling control apparatus 10 has a sequence modification function 173, as shown in FIG. 7. By implementing the sequence modification function 173, the processing circuitry 17 accepts a modification made by a user to the content of the determined sequence. At this time, the processing circuitry 17 causes a display of the output interface 15 to display the selected sequence based on the acquired control token. The user can determine whether or not to adopt the selected sequence. Also, by implementing the sequence modification function 173, the processing circuitry 17 accepts a modification made by a user to the content of a sequence. The user can appropriately modify the content of a sequence via the input interface 16. Multiple sequences may be determined as candidates to be adopted and presented to the user. In this case, the user can select an appropriate sequence from among the multiple sequence candidates displayed.

### (Other Modifications)

In the above embodiment, a sequence is selected according to the types of cell handling devices 20 that perform different types of processes; however, even if the same type of process is to be performed, a sequence to be selected may be changed according to the conditions of the processes. For example, control parameters such as a liquid delivery rate and a liquid delivery timing may be adjusted according to the type, amount, and viscosity of a liquid such as a cell fluid or a reagent set in the cell handling device 20. For example, in this case, a bar-code including information relating to the type, amount, and viscosity of a liquid is used as a control token. Alternatively, the processing circuitry 17 may analyze the amount, viscosity, and color of a liquid such as blood or a reagent using a spectral analysis. In this case, an observation window allowing for observation of a liquid inside the cell handling device 20 may be provided as the control token expression unit 21.

The control token may also include sequence information including operation timing of each device of the auxiliary mechanism 11, an operation order, and a control parameter. In this case, the cell handling control apparatus 10 performs cell handling according to the sequence information included in the acquired control token.

Also, the cell handling control apparatus 10 may collate the acquired control token with a preset control token and determine whether or not the acquired identification information is preauthenticated. For example, if the received control token is a preauthenticated control token, the cell handling control apparatus 10 determines that the attached device is an authenticated cell handling device 20, and performs cell handling according to the selected sequence. On the other hand, if the acquired control token is not a preauthenticated control token, the cell handling control apparatus 10 determines that the attached device is not an authenticated cell handling device 20, stops cell handling while it is being performed, and warns a user. Also, if the control token includes sequence information, whether or not the cell handling process performed according to the sequence information included in the received control token is an authenticated cell handling process may be recorded. A storage medium capable of recording information indicating whether the performed cell handling process is an authenticated cell handling process or not may be provided in the cell handling device 20.

The cell handling device 20 may have a function of supporting the function fulfilled by the control execution function 172. For example, a function of supporting liquid delivery, a function of supporting adjustment of a culturing environment, or a function of supporting observation may be performed by processing circuitry or a drive mechanism provided to the cell handling device 20. The function of supporting liquid delivery includes, for example, controlling container inclination to facilitate liquid delivery. The function of supporting adjustment of a culturing environment includes, for example, controlling a vent to control temperature adjustment. The function of supporting observation includes, for example, controlling the position of an observation device, an observation window, an observation mirror, etc.

The terminology “processor” used in the above description refers to, for example, circuitry such as a CPU (central processing unit), a GPU (graphics processing unit), an ASIC (application specific integrated circuit), a programmable logic device (such as a simple programmable logic device (SPLD), a complex programmable logic device (CPLD), and a field programmable gate array (FPGA)), etc. If the processor is, for example, a CPU, the processor implements the functions by reading and executing programs stored in the storage circuitry 13. On the other hand, if the processor is an ASIC, its functions are directly incorporated into the circuitry of the processor as logic circuitry, instead of a program being stored in the storage circuitry 13. Each processor of the present embodiment is not limited to be configured as single circuitry; multiple sets of independent circuitry may be integrated into a single processor that implements its functions. Furthermore, multiple components may be integrated into a single processor to implement the functions of the processor.

In the above embodiment, descriptions will be given on the premise that each function is implemented by a single processor; however, the embodiment is not limited thereto. For example, a plurality of independent processors may be combined to constitute control circuitry, and the respective processors may implement the respective functions by executing the programs. Each function may be installed as individual hardware circuitry. The processing circuitry 17 may include a storage area for storing at least part of the data stored in the storage circuitry 13.

According to at least one embodiment explained above, the cell handling system that can use multiple cell handling devices capable of performing different processes relating to cell handling allows for reductions in production costs, user time and user effort.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell handling system comprising
a cell handling device configured so that cell handling is performed inside the cell handling device, the cell handling device comprising an information presenter configured to present identification information for identifying a process performed in the cell handling device and
a cell handling control apparatus configured to control the cell handling performed in the cell handling device, the cell handling control apparatus comprising processing circuitry configured to acquire the identification information presented by the information presenter and determine a parameter relating to control of the cell handling based on the acquired identification information.

2. The cell handling system according to claim 1,
wherein the processing circuitry determines the parameter relating to control of the cell handling by determining a sequence including multiple processes based on the identification information.

3. The cell handling system according to claim 2,
further comprising a storage that stores multiple sequences,
wherein the processing circuitry specifies a type of the cell handling device based on the identification information and selects, from among the sequences stored in the storage, a sequence corresponding to the specified type.

4. The cell handling system according to claim 1,
wherein the processing circuitry comprises an auxiliary mechanism configured to assist the cell handling performed in the cell handling device and controls an operation of the auxiliary mechanism based on the parameter relating to control.

5. The cell handling system according to claim 4, wherein
the cell handling includes at least one of cell processing or cell culturing, and
the auxiliary mechanism includes at least one of a liquid delivery mechanism, a temperature adjustment mechanism, or an observation mechanism.

6. The cell handling system according to claim 4,
wherein the processing circuitry controls the operation of the auxiliary mechanism by performing direct control, period control, or information acquisition control on the auxiliary mechanism.

7. The cell handling system according to claim 1, wherein
the cell handling device comprises a record transmitter configured to transmit execution information relating to an actually performed process, and
the processing circuitry records the execution information acquired from the record transmitter.

8. The cell handling system according to claim 2,
wherein the processing circuitry accepts a modification made to the determined sequence.

9. A cell handling device configured so that cell handling is performed therein,
the cell handling device comprising an information presenter configured to present, to a cell handling control apparatus configured to control the cell handling, identification information for identifying a process performed in the cell handling device.

10. A cell handling control apparatus configured to control cell handling performed in a cell handling device,
the cell handling control apparatus comprising processing circuitry configured to acquire, from the cell handling device, identification information for identifying a process performed in the cell handling device and determine a parameter relating to control of the cell handling based on the acquired identification information.
